(19) United States
(12) Patent Application Publication
(10) Pub. No.: US 2022/0192627 A1
(43) Pub. Date: Jun. 23, 2022

(54) STRESS ECHOCARDIOGRAM IMAGING COMPARISON TOOL

(71) Applicant: INTERNATIONAL BUSINESS MACHINES CORPORATION, Armonk, NY (US)

(72) Inventors: Jennifer C. Quattrone-Brown, Garland, TX (US); Mark D. Bronkalla, Waukesha, WI (US)

(21) Appl. No.: 17/131,949

(22) Filed: Dec. 23, 2020

(51) Int. Cl. A61B 8/08; A61B 5/00; A61B 8/00; A61B 8/14

(52) U.S. Cl. CPC A61B 8/0883; A61B 5/4884; A61B 8/463; A61B 8/488; A61B 8/5284; A61B 5/7267; A61B 8/42; A61B 8/14

(57) ABSTRACT

An embodiment of the invention may include a method, computer program product and system for analyzing cardiac function of a patient. An embodiment may include receiving a plurality of digital image representations of cardiac function at rest. An embodiment may include receiving a plurality of digital image representations of cardiac function at stress. An embodiment may include selecting a digital image representation of cardiac function at rest and a corresponding digital image representation of cardiac function at stress. An embodiment may include aligning the selected representation of cardiac function at rest with the selected corresponding representation of cardiac function at stress. An embodiment may include identifying a difference between the selected representation of cardiac function at rest and the selected corresponding representation of cardiac function at stress based on a displayed overlay of the aligned representations.

### BACKGROUND

Embodiments of the present invention relate generally to the field of image processing, more specifically to image processing in medical diagnostics.

Medical imaging is the technique and process of creating visual representations of the interior of a body for clinical analysis and medical intervention, as well as visual representation of the function of some organs or tissues. Medical imaging seeks to reveal internal structures hidden by the skin and bones, as well as to diagnose and treat disease, such as coronary artery disease. Medical imaging also establishes a database of normal anatomy and physiology to make it possible to identify abnormalities. As of 2010, 5 billion medical imaging studies had been conducted worldwide.

### BRIEF SUMMARY

An embodiment of the invention may include a method, computer program product and system for analyzing cardiac function of a patient. An embodiment may include receiving a plurality of digital image representations of cardiac function at rest. An embodiment may include receiving a plurality of digital image representations of cardiac function at stress. An embodiment may include selecting a digital image representation of cardiac function at rest and a corresponding digital image representation of cardiac function at stress. An embodiment may include aligning the selected representation of cardiac function at rest with the selected corresponding representation of cardiac function at stress. An embodiment may include identifying a difference between the selected representation of cardiac function at rest and the selected corresponding representation of cardiac function at stress based on a displayed overlay of the aligned representations.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1 is a block diagram illustrating a Myocardium Contraction Detection (MCD) System, in accordance with an embodiment of the present invention;

FIGS. 2A-2C depict ultrasound images of myocardium at mitral level in sagittal view, in accordance with an embodiment of the present invention;

FIG. 3 depicts an ultrasound image of the heart indicating tissue doppler speeds for use in tissue delineation, in accordance with an embodiment of the present invention;

FIG. 4 is a flowchart illustrating the operations of the Myocardial Image Analysis Module of FIG. 1, in accordance with an embodiment of the invention;

FIG. 5 is a block diagram depicting the hardware components of the MCD System of FIG. 1, in accordance with an embodiment of the invention;

FIG. 6 depicts a cloud computing environment in accordance with an embodiment of the present invention; and

FIG. 7 depicts abstraction model layers in accordance with an embodiment of the present invention.

### DETAILED DESCRIPTION

Coronary artery disease (CAD), also known as coronary heart disease (CHD), ischemic heart disease (IHD), or simply heart disease, involves the reduction of blood flow to the heart muscle due to a build-up of plaque in the arteries of the heart. Coronary artery disease is the most common of the cardiovascular diseases. In 2015, coronary artery disease affected 110 million people and resulted in 8.9 million deaths. Coronary artery disease makes up 15.6% of all deaths, making it the most common cause of death globally. In the United States, cardiovascular disease is the number one and number three cause of death, accounting for more deaths than all cancers combined.

CAD is the most common cause of coronary ischemia. Coronary ischemia, myocardial ischemia, or cardiac ischemia is a medical term for reduced blood flow circulation through the coronary arteries. Coronary circulation is the circulation of blood in the blood vessels that supply the heart muscle, also known as the myocardium. As coronary arteries deliver oxygen-rich blood to the heart muscle, reduced blood flow to the heart associated with coronary ischemia can result in an inadequate oxygen supply to the heart muscle. When oxygen supply to the heart does not meet the oxygen demand from the myocardium, the result includes the characteristic symptoms of coronary ischemia, the most common of which is chest pain. Moreover, if blood flow through the coronary arteries is stopped completely, cardiac muscle cells may die and result in a myocardial infarction, also known as a heart attack. If coronary ischemia is suspected, a series of diagnostic tests may be undertaken for confirmation and diagnoses. The most common diagnostic tests used are an electrocardiogram, an exercise stress electrocardiogram, and stress echocardiography.

A resting electrocardiogram is an early step in the diagnostic process. An electrocardiogram (ECG) is a graph of voltage versus time of the electrical activity of the heart detected using electrodes placed on the skin. These electrodes detect the small electrical changes that are a consequence of cardiac muscle depolarization followed by repolarization during each cardiac cycle (heartbeat). The cardiac cycle is the performance of the human heart from the ending of one heartbeat to the beginning of the next heartbeat (NHB). It consists of two periods: one during which the heart muscle relaxes and refills with blood, called diastole, following a period of robust contraction and pumping of blood, dubbed systole. Changes in the normal ECG pattern occur in numerous cardiac abnormalities, including inadequate coronary artery blood flow, such as myocardial ischemia and myocardial infarction. There are three main components to an ECG readout: the P-wave, which represents the depolarization of the atria; the QRS complex, which represents the depolarization of the ventricles; and the T wave, which represents the repolarization of the ventricles. To a trained clinician, an ECG conveys a large amount of information about the structure of the heart and the function of its electrical conduction system. Among other things, an ECG can be used to measure the rate and rhythm of heartbeats, the size and position of the heart chambers, and the presence of any damage to the heart's muscle cells or conduction system.

An exercise stress electrocardiogram, also known as a cardiac stress test, uses an ECG to detect the electrical impulses of the heart during physical exertion. The stress response is commonly induced through exercise on a treadmill or pedaling a stationary exercise bicycle. Cardiac stress tests compare the coronary circulation while the patient is at rest with the same patient's circulation during maximum cardiac exertion, showing any abnormal blood flow to the myocardium. This test can be used to diagnose coronary artery disease and assess patient prognosis after a myocardial infarction. However, an exercise stress electrocardiogram is not always accurate in determining the presence of a blockage in the arteries.

In addition to an electrocardiogram, stress echocardiography is commonly used in detecting ischemia resulting from CAD. The echocardiography is performed both before and after the stress (e.g. exercise) so that structural differences can be compared. A stress echocardiogram, also known as a stress echo, uses ultrasound imaging of the heart to assess the wall motion in response to physical stress. First, images of the heart are taken “at rest” to acquire a baseline of the patient's wall motion at a resting heart rate. The patient then walks on a treadmill or uses another exercise modality to increase the heart rate to a target heart rate. Finally, images of the heart are taken “at stress” to assess wall motion at the peak heart rate. Ischemia of one or more coronary arteries can be detected by visualizing abnormalities in the movement and thickness of the heart wall during exercise. During both the rest and stress portions of the stress echo, ECG readings of the patient may also be recorded. Additionally, an echocardiogram can also produce an accurate assessment of the blood flowing through the heart via doppler echocardiography, using pulsed or continuous-wave doppler ultrasound. This allows assessment of both normal and abnormal blood flow through the heart. Blood flow is often seen as color images (i.e., using doppler color flow imaging) flowing through the heart on a monitor connected to the ultrasound machine. The doppler technique can also be used for tissue motion and velocity measurement, through tissue doppler imaging (TDI).

TDI has been used in deriving myocardial velocities and assessing fundamental parameters of myocardial deformation (strain and strain rate) in addition to providing insights into ischemia, myocardial mechanics and many other processes of the heart. Another known imaging modality which permits calculation of myocardial velocities and deformation parameters such as strain and strain rate is two-dimensional (2D) speckle tracking echocardiography (STE). STE is based on 2D echocardiographic technology in which segments of myocardial tissue show a pattern, called a speckle pattern, of gray values in the ultrasound. This speckle pattern, resulting from the spatial distribution of gray values, characterizes the underlying myocardial tissue acoustically and is unique for each myocardial segment. Myocardial segments are the anatomical units of myocardium for which results of strain analysis will be reported. STE allows the measure of all in-plane components of the velocity vector, in all pixels.

Notwithstanding the aforementioned known diagnostic tests and imaging modalities currently in practice for assessment of cardiovascular disease, global myocardial ischemia and coronary artery disease of less than seventy percent arterial blockage often go undetected. Consequently, a large segment of the population is left at risk for heart disease before ever repeating a diagnostic test which detects a sufficient ischemia or arterial blockage for a diagnosis of cardiovascular disease. Through providing both visual and quantifiable information of detected cardiovascular disease which may not readily present during traditional diagnostic tests, embodiments of the invention propose a Myocardium Contraction Detection (MCD) System 100 to adjust the risk stratification of patients who potentially have had previously undetected cardiovascular disease by detecting discrete changes in myocardial wall motion which may be indicative of global ischemia. As risk stratification uses a mix of objective and subjective data to assign risk levels to patients and determine an appropriate level of medical care, having a more accurately adjusted risk stratification for patients, who potentially have had previously undetected cardiovascular disease, can be lifesaving. Furthermore, implementation of MCD System 100 may result in tremendous patient care cost savings worldwide. The World Health

Organization estimates that 32.4 million people suffer from CAD every year. Of that 32.4 million people, the National Institute for Health estimates that 0.024 of all CAD patients will be subject to global ischemia with an average care cost of $15,000.00 USD (when a cardiac event occurs). As such, the early detection of myocardial ischemia made possible by MCD System 100 could result in a potential patient care cost savings of $12,104,145,000.00 USD globally.

In embodiments of the invention, MCD System 100 performs a comparison of at rest and at stress representations of cardiac function (i.e., myocardium ultrasound images at rest and at stress) of a patient, acquired during a stress echocardiogram, and visually identifies potential areas of myocardial ischemia. Initially, a technician, or other qualified medical professional, takes cardiac images in 2D resting protocol (i.e., at rest) to create a number of reference images of the patient's myocardium via echocardiography. This resting protocol contains all of the standard images needed for comparison by MCD System 100. During this step, the technician marks the most optimal images to be used as a guide during the acquisition of images at stress. During stress protocol imaging, MCD System 100 prompts the technician, based on the previously acquired resting protocol images, to adjust the angle of ultrasound probe in order to acquire an image that aligns for comparison, by MCD System 100, of rest and stress myocardium images. In embodiments of the invention, the patient will also be undergoing an ECG during the stress echo, as such, MCD System 100 aligns the rest and stress images using the R-wave of the ECG for synchronization. The R-wave is the first upward deflection after the P-wave and provides information on the timing of cardiac cycles. The purpose of the comparison is to view the difference in response of segmental myocardial tissue in order to determine the lack of or the presence of myocardial ischemia. Furthermore, the comparison would allow for the possible illumination of discrete changes, as may be seen in global ischemia, thereby raising the sensitivity and precision of a stress echo. In embodiments of the invention, all image processing by MCD System 100 is performed on a U.S. workstation and then sent in the digital imaging and communications in medicine (DICOM) format to a cardiology picture archiving and communication system (CPACS) for review.

Embodiments of the present invention will now be described in detail with reference to the accompanying Figures.

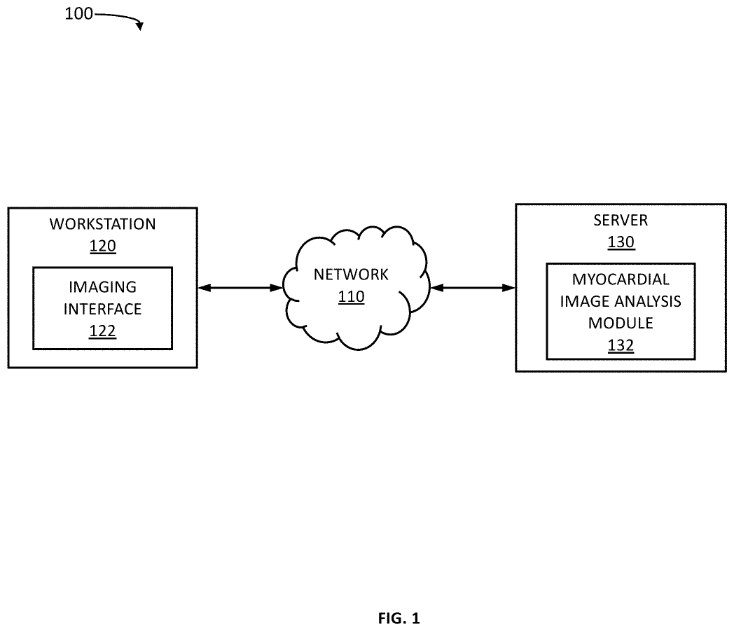

FIG. 1 is a functional block diagram illustrating Myocardium Contraction Detection (MCD) System 100, in accordance with an embodiment of the present invention. MCD System 100 may be a network of computers, medical imaging devices, and data processing systems in which the illustrative embodiments may be implemented. In an example embodiment, MCD System 100 may include workstation 120, and server 130, all interconnected via network 110. In an example embodiment, MCD System 100 may also include one or more cardiac diagnostic devices (not shown) which send and receive data to and from workstation 120 and/or server 130.

In various embodiments, network 110 is a communication channel capable of transferring data between connected devices. In an example embodiment, network 110 may be the Internet, representing a worldwide collection of networks and gateways to support communications between devices connected to the Internet. Moreover, network 110 may include, for example, wired, wireless, or fiber optic connections which may be implemented as an intranet network, a local area network (LAN), a wide area network (WAN), or any combination thereof. In further embodiments, network 110 may be a Bluetooth network, a WiFi network, or a combination thereof. In general, network 110 can be any combination of connections and protocols that will support a network of computers, medical imaging devices, and data processing systems and may support communications between workstation 120 and server 130.

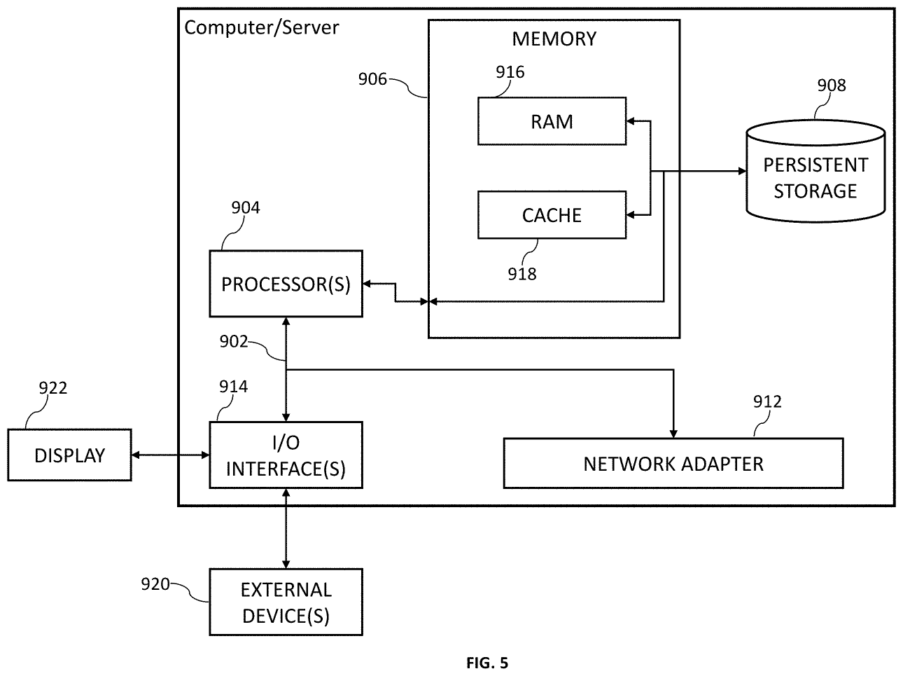

In an example embodiment, workstation 120 may include imaging interface 122. Workstation 120 may be a laptop computer, a notebook, a tablet computer, a netbook computer, a personal computer (PC), a desktop computer, or any other electronic device or computing system capable of sending, and receiving data to and from other computing devices such as cardiac diagnostic devices (not shown) (e.g., an ultrasound machine) and server 130, via network 110, and capable of supporting the functionality required of embodiments of the invention (e.g., receiving and/or directing the acquisition of representations of cardiac functions for analysis via MCD System 100). For example, workstation 120 may support a communication link (e.g., wired, wireless, direct, via a LAN, via the network 110, etc.) between workstation 120, cardiac diagnostic devices (not shown), and server 140. Data sent from workstation 120 may include data from imaging interface 122. Data received by workstation 120 may include data sent, via network 110, from one or more cardiac diagnostic devices (not shown) and/or server 130. Additionally, a cardiac diagnostic device (not shown) of MCD System 100 may be attached to a post image/video capture device (not shown) for digital and/or analog conversion of images/video acquired by the cardiac diagnostic device. Workstation 120 may be described, generally, with respect to FIG. 5 below. In an example embodiment, workstation 120 may send, via network 110, data captured by imaging interface 122 (e.g., representations of cardiac functions received from one or more cardiac diagnostic devices) to myocardial image analysis module 132 located on server 130. In an example embodiment, workstation 120 may also receive data from myocardial image analysis module 132 located on server 130.

In an example embodiment, imaging interface 122 may be a program, or subroutine contained in a program, that may operate to receive and display one or more representations of cardiac functions (e.g., myocardial images, at rest and at stress, resulting from a stress echo of a patient and a corresponding ECG readout resulting from an electrocardiogram of the patient during the stress echo) from one or more cardiac diagnostic devices, interact with myocardial image analysis module 132 located on server 130, via network 110, and present cardiac function representation (e.g., cardiac image) analysis results from myocardial image analysis module 132 to a user (e.g., a medical technician) of workstation 120. In an example embodiment, imaging interface 122 may be a user interface for a cardiac diagnostic device acquiring images of a patient's heart via ultrasound (e.g., echocardiography). For example, imaging interface 122 might prompt, based on input from myocardial image analysis module 132, a medical technician performing an echocardiogram of a patient on how to position the ultrasound probe so as to acquire particular images. In addition, imaging interface 122 may be connectively coupled to hardware components, such as those depicted by FIG. 5, for receiving user input, including mice, keyboards, touchscreens, microphones, cameras, and the like. In embodiments, imaging interface 122 may be implemented via other integrated or standalone commercially available, open source, or proprietary software applications and hardware capable of receiving user interaction and communicating with other electronic devices. Additionally, in an example embodiment, imaging interface 122 may send and receive data (e.g., cardiac function representations/images of a patient, analysis of cardiac function representations/images of a patient) to and from server 130, via network 110.

In an example embodiment, server 130 may include myocardial image analysis module 132. Server 130 may be a desktop computer, a notebook, a laptop computer, a blade server, a networked computer appliance, a virtual device, or any other networked electronic device or computing system capable of receiving and sending data from and to other computing devices such as workstation 120, via network 110, and capable of supporting the functionality required of embodiments of the invention. In embodiments of the invention, server 130 may host a cardiac representation/image analysis application (e.g., myocardial image analysis module 132) for comparison and analysis of cardiac representations (e.g., myocardial images and ECG readouts) of a person. In an example embodiment, server 130 may function to process data received from workstation 120, via network 110. While server 130 is shown as a single device, in other embodiments, server 130 may represent a cluster or plurality of servers, working together or working separately. Server 130 may be described generally with respect to FIG. 5 below.

In an example embodiment, myocardial image analysis module 132 may be a program, or subroutine contained in a program, that may receive cardiac representations of a patient, both at rest and at stress, perform a comparison/analysis of the received cardiac representations, and visually identify potential areas of myocardial ischemia based on the comparison/analysis of the received cardiac representations. The received cardiac representations may include digital myocardial ultrasound images of a patient at rest, as well as at stress, and may include a corresponding ECG readout of the patient both at rest and at stress. Moreover, the myocardial ultrasound images may be taken at mitral level (i.e., the ultrasound probe may be positioned across the mitral valve of the heart) in a sagittal plane view. In an example embodiment, myocardial image analysis module 132 may overlay a received cardiac representation at rest with a received corresponding cardiac representation at stress in order to visually identify one or more areas of cardiac tissue that is not contracting concentrically.

The clinical significance of such an identification is that the non-concentrically contracting areas of cardiac tissue may be areas affected by cardiac ischemia. In an example embodiment, myocardial image analysis module 132 may align a cardiac representation at rest and a corresponding cardiac representation at stress using the R-wave in an ECG readout for synchronization between the representations. Additionally, myocardial image analysis module 132 may utilize tissue doppler speeds, resulting from cardiac TDI of the patient, and/or STE in aligning the cardiac representation at rest and the corresponding cardiac representation at stress. Furthermore, in an example embodiment, myocardial image analysis module 132 may prompt, in real time, a medical ultrasound technician to adjust positioning of an ultrasound probe based on the information used in aligning cardiac representations (e.g., ECG R-wave, tissue doppler speeds, and/or STE), in order to acquire the optimal cardiac representations at stress for comparison with the corresponding cardiac representations at rest.

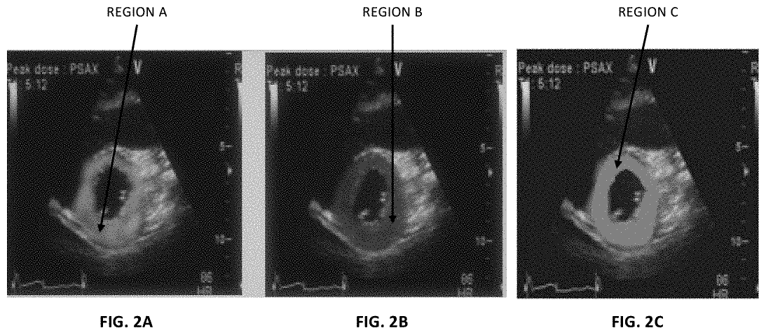

FIGS. 2A-2C depict digital ultrasound images of myocardium at mitral level in sagittal view, in accordance with an example embodiment of the invention. The images depicted in FIGS. 2A-2C are examples of the kinds of representations of cardiac function which are compared and analyzed by MCD System 100. In FIG. 2A, Region A in yellow depicts at rest myocardium at mitral level in sagittal view. In FIG. 2B, Region B in blue depicts at stress myocardium at mitral level in sagittal view. FIG. 2C depicts the overlay of Region A, of FIG. 2A, and Region B, of FIG. 2B, highlighting the portion of the myocardium that is not contracting concentrically in yellow outside of Region C in green which depicts both the at rest and at stress responsive regions of the myocardium. In an example embodiment, myocardial image analysis module 132 may receive images such as those depicted in FIGS. 2A and 2B, apply image coloration to differentiate between the at rest and at stress myocardium response in the received images, and produce an image such as FIG. 2C which visualizes, through an overlay of the at rest image with the at stress image, the difference (i.e., the yellow portion outside of the green portion) in the myocardial wall motion (i.e., contraction). Furthermore, in an example embodiment, myocardial image analysis module 132 may align images such as FIGS. 2A and 2B for overlay (thus producing an image such as FIG. 2C) using a corresponding ECG of the myocardium represented in FIGS. 2A and 2B to synchronize on the R-wave and speckle tracking echocardiography in the pixels of the digital images displayed on a monitor for alignment.

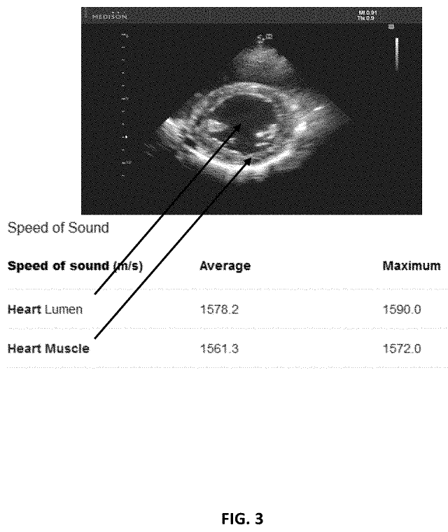

FIG. 3 depicts an ultrasound image of the heart indicating tissue doppler speeds for use in tissue delineation, in accordance with an example embodiment of the invention. In an example embodiment, myocardial image analysis module 132 may use tissue doppler speeds to identify and delineate the inner and outer lumen (the inside space of a tubular structure) of the heart in order to identify matching image frames between an image of at rest myocardium (e.g. FIG. 2A) and an image of at stress myocardium (e.g., FIG. 2B), and thus determine the location where comparison/analysis of the images (e.g., FIG. 2C) should take place. As FIG. 3 illustrates, delineation is made possible as the speed of sound is, on average, slower within the outer lumen of the heart (e.g., myocardium) as compared with the inner lumen of the heart. It should be noted that in cases of pericardial effusion, plural effusion, and cardiac amyloidosis, the use of tissue doppler speeds to identify and delineate the inner and outer lumen of the heart is not recommended due to additional echogenic areas associated with these cases.

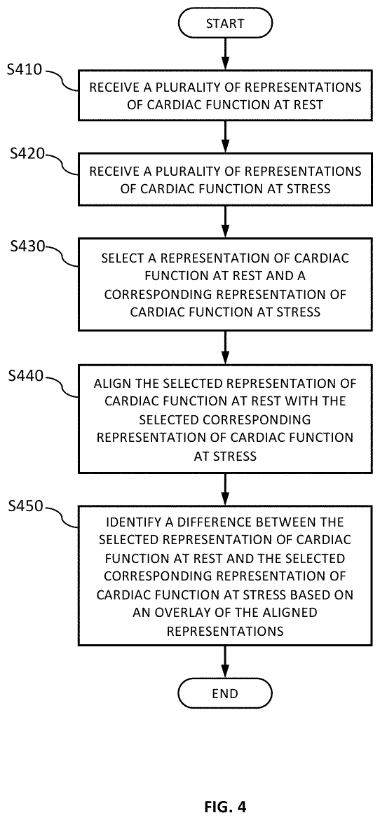

FIG. 4 shows a flowchart illustrating the operations of myocardial image analysis module 132 in accordance with an example embodiment of the invention. Referring to step S410, myocardial image analysis module 132 receives a plurality of digital image representations of cardiac function at rest for at least one cardiac cycle. In an example embodiment, the received plurality of cardiac representations at rest may include a plurality of digital ultrasound images of one or more cardiac cycles of a patient's heart at rest. The plurality of ultrasound images may be acquired by a medical technician during an initial rest phase of a stress echocardiogram of the patient. Furthermore, the plurality of ultrasound images may be acquired by an ultrasound probe positioned, by the technician, at mitral level and may show a sagittal and/or transverse anatomical view. In an example embodiment, at least one image of the received plurality of ultrasound images of one or more cardiac cycles of the patient's heart at rest may be flagged for use in correlation to at least one ultrasound image of the patient's heart at stress acquired by the medical technician during a later phase of the stress echocardiogram of the patient. The flagging may be based on an optimal/desired angle or orientation of the at least one image (resulting from positioning of the ultrasound probe), an optimal/desired anatomical view of the at least one image, and/or an optimal/desired clarity of the at least one image. In an example embodiment, the plurality of ultrasound images may be received from a workstation connected to a medical diagnostic device.

Referring to step S420, myocardial image analysis module 132 receives a plurality of representations of cardiac function at stress for at least one cardiac cycle. In an example embodiment, the received plurality of cardiac digital image representations at stress may include a plurality of digital ultrasound images of one or more cardiac cycles of a patient's heart at stress. The plurality of ultrasound images may be acquired by a medical technician during a stress phase of the stress echocardiogram of the patient and acquired within 30 seconds of the stress inducing activity. Furthermore, the plurality of ultrasound images may be acquired by an ultrasound probe positioned, by the technician, at mitral level and may show a sagittal and/or transverse anatomical view. Additionally, in an example embodiment, myocardial image analysis module 132 may prompt, in real time, the medical technician performing the stress echocardiogram of the patient during the stress phase on how to position the ultrasound probe so as to acquire at least one digital ultrasound image of the patient's heart at stress for correlation and/or comparison to at least one digital ultrasound image of the patient's heart at rest previously acquired during the stress echocardiogram. The prompting by myocardial image analysis module 132 may be based on an angle or orientation, an anatomical view, and/or a clarity of the at least one flagged ultrasound image of the patient's heart at rest. Moreover, the prompting may be via text and/or directional arrow(s) displayed on a monitor attached to the medical device acquiring the ultrasound images of the patient's heart during the stress echocardiogram. The prompting may also be provided via audio instructions played though a speaker of the medical device acquiring the ultrasound images, or a speaker of a workstation connected to the medical device acquiring the ultrasound images. The at least one ultrasound image of the patient's heart at stress acquired as a result of prompting by myocardial image analysis module 132 may be included in the received plurality of ultrasound images of one or more cardiac cycles of a patient's heart at stress. In an example embodiment, the plurality of ultrasound images may be received from a workstation connected to a medical diagnostic device.

Referring to step S430, myocardial image analysis module 132 selects a digital image representation of cardiac function at rest and a corresponding digital image representation of cardiac function at stress. In embodiments of the invention, the plurality of digital image representations of cardiac function at rest received in step S410 and the plurality of digital image representations of cardiac function at stress received in step S420 are respectively ranked, by myocardial image analysis module 132, across one or more cardiac cycles from most to least desirable based on representation orientation resulting from ultrasound probe orientation and angle, anatomical view (e.g., sagittal vs. transverse), representation clarity, and/or diagnostic quality as it pertains to a view of the myocardium function having clear pixels and anatomical landmarks to match between at rest and at stress states of the myocardium. In embodiments of the invention, myocardial image analysis module 132 may rank the received representations using a machine learning model trained on optimal cardiac representations acquired from a plurality of patients across a variety of demographics. A cardiac representation may be considered optimal when it is determined to display a desired orientation/angle, anatomical view, pixel clarity, and/or diagnostic quality in accordance with ultrasound imaging best practices defined within the medical community. In an example embodiment, myocardial image analysis module 132 selects, within a given cardiac cycle, at least one representation of cardiac function at rest and at least one representation of cardiac function at stress having some degree of matching representation orientation resulting from ultrasound probe orientation and angle, anatomical view (e.g., sagittal vs. transverse), representation clarity, and/or diagnostic quality as it pertains to a view of the myocardium function having clear pixels and anatomical landmarks to match between at rest and at stress states of the myocardium. The selected representation of cardiac function at rest and the selected corresponding representation of cardiac function at stress may be representations having a highest degree of match. Alternatively, myocardial image analysis module 132 may select the representation of cardiac function at rest and the corresponding representation of cardiac function at stress based on a ranking for each being most desirable or based on a ranking for each being above a threshold level. In an example embodiment, myocardial image analysis module 132 selects an ultrasound image of the received plurality of ultrasound images of one or more cardiac cycles of the patient's heart at rest, and selects a corresponding ultrasound image of the received plurality of ultrasound images of one or more cardiac cycles of a patient's heart at stress based on the images having a highest degree of match.

It should be noted that in another embodiment, myocardial image analysis module 132 may receive a second plurality of representations of cardiac function at rest for at least one cardiac cycle. This received second plurality of representations may be acquired subsequent to the stress phase of the same stress echocardiogram when the patient's heart function has returned to a resting state. Furthermore, this second plurality of representations may be acquired by an ultrasound probe positioned, by the technician, at mitral level and may show a sagittal and/or transverse anatomical view. At least one image of the received second plurality of representations may be ranked and selected, as described above in step S430, for use in correlation/comparison to at least one ultrasound image of the patient's heart at stress acquired by the medical technician during the previously executed stress phase of the stress echocardiogram of the patient. Moreover, the technician may be prompted, by myocardial image analysis module 132, on how to position the ultrasound probe so as to acquire at least one ultrasound image of the patient's heart at rest for correlation and/or comparison to at least one ultrasound image of the patient's heart at stress previously acquired during the stress echocardiogram. The prompting by myocardial image analysis module 132 may be based on an angle or orientation, an anatomical view, and/or a clarity of at least one flagged ultrasound image of the patient's heart at stress.

Referring to step S440, myocardial image analysis module 132 aligns the selected representation of cardiac function at rest with the selected corresponding representation of cardiac function at stress. As part of the alignment process, myocardial image analysis module 132 may delineate, via the use of tissue doppler imaging and observed tissue doppler speeds during the stress echo of the patient, between the myocardium and the surrounding tissue and segment appropriately by endocardium (inner wall) and epicardium (outer wall) in order to detect the endocardial and epicardial contours of the patient's heart. Using the endocardial and epicardial contours as references, myocardial image analysis module 132 may align the selected representation of cardiac function at rest and the selected representation of cardiac function at stress at each portion of a cardiac cycle. Additionally, myocardial image analysis module 132 may align the selected representation of cardiac function at rest and the selected corresponding representation of cardiac function at stress using a corresponding ECG of the patient (taken during the stress echo) to synchronize on the R-wave of the ECG and using speckle tracking echocardiography in the pixels of the selected representations displayed on a monitor for alignment. Furthermore, myocardial image analysis module 132 may utilize color coding within pixels of the digital images, as depicted in FIGS. 2A-2C, to differentiate between the at rest response and the at stress response (e.g., difference in wall motion of the heart) of the selected representation of cardiac function at rest and the selected representation of cardiac function at stress, respectively. In an example embodiment, myocardial image analysis module 132 aligns the selected ultrasound image of the received plurality of ultrasound images of one or more cardiac cycles of the patient's heart at rest, and the selected corresponding ultrasound image of the received plurality of ultrasound images of one or more cardiac cycles of a patient's heart at stress using detected the endocardial and epicardial contours of the patient's heart (delineated via the use of tissue doppler imaging and observed tissue doppler speeds), a corresponding ECG of the patient to synchronize on the R-wave, and/or speckle tracking echocardiography in the pixels of the selected ultrasound images displayed on a monitor for alignment

Referring to step S450, myocardial image analysis module 132 identifies a difference between the selected representation of cardiac function at rest and the selected corresponding representation of cardiac function at stress based on a displayed overlay of the aligned representations. Myocardial image analysis module 132 may create a color coded fused image, such as that depicted in FIG. 2C, by overlaying, on a monitor, the selected representation of cardiac function at rest with the selected corresponding representation of cardiac function at stress. As a result of the color coding in step S440, myocardial image analysis module 132 can identify, highlight and display to a medical professional one or more portions of the patient's myocardium that is not contracting concentrically. For example, if the selected representation of cardiac function at rest is color coded in yellow and the selected corresponding representation of cardiac function at stress is color coded in blue, an overlay of the selected representations may display a portion of the myocardium color coded in green which depicts both the at rest and at stress responsive regions of the myocardium (e.g., the portions of the myocardium wall contracting concentrically) and potentially one or more portions of the myocardium color coded in yellow outside of the green portion. The clinical significance of visually identifying any portions of the myocardium color coded in yellow outside of the green portion is that the technician (or another medical professional) may determine that these yellow portions of the myocardium are not contracting concentrically possibly as a result of local or global myocardial ischemia. The ability of myocardial image analysis module 132 to detect the possibility of global ischemia and native vessel disease of less than seventy percent arterial blockage may greatly improve patient outcomes in treating CAD. In an example embodiment, myocardial image analysis module 132 overlays the aligned ultrasound images of step S440 and visually identifies (using image coloration) to a medical professional one or more portions of the patient's myocardium wall that is not contracting concentrically possibly due to a lack of blood flow or ischemic deficit.

In an alternate embodiment, myocardial image analysis module 132 may utilize the Sorensen-Dice coefficient in addition to the Hausdorff distance to determine if a potential risk of ischemia is depicted within the representations. The Sorensen-Dice coefficient is a statistic used to gauge the similarity of two samples. In the context of MCD System 100, one sample may be the selected representation of cardiac function at rest and the other sample may be the selected corresponding representation of cardiac function at stress. The Hausdorff distance will detect shape irregularities that the Sorensen-Dice coefficient may ignore, thus enabling automated detection, by myocardial image analysis module 132, of a potential risk of ischemia despite the presence of issues such as false aneurysm or other myocardial wall motion irregularities. Furthermore, in an alternate embodiment, the selected representation of cardiac function at rest, the selected corresponding representation of cardiac function at stress, the overlay of the selected representations may be displayed in motion.

FIG. 5 depicts a block diagram of components of workstation 120 and server 130, in accordance with an illustrative embodiment of the present invention. It should be appreciated that FIG. 5 provides only an illustration of one implementation and does not imply any limitations with regard to the environments in which different embodiments may be implemented. Many modifications to the depicted environment may be made.

Workstation 120 and server 130 include communications fabric 902, which provides communications between computer processor(s) 904, memory 906, persistent storage 908, network adapter 912, and input/output (I/O) interface(s) 914. Communications fabric 902 can be implemented with any architecture designed for passing data and/or control information between processors (such as microprocessors, communications and network processors, etc.), system memory, peripheral devices, and any other hardware components within a system. For example, communications fabric 902 can be implemented with one or more buses.

Memory 906 and persistent storage 908 are computer-readable storage media. In this embodiment, memory 906 includes random access memory (RAM) 916 and cache memory 918. In general, memory 906 can include any suitable volatile or non-volatile computer-readable storage media.

The programs imaging interface 122 in workstation 120 and myocardial image analysis module 132 in server 130 are stored in persistent storage 908 for execution by one or more of the respective computer processor(s) 904 via one or more memories of memory 906. In this embodiment, persistent storage 908 includes a magnetic hard disk drive. Alternatively, or in addition to a magnetic hard disk drive, persistent storage 908 can include a solid state hard drive, a semiconductor storage device, read-only memory (ROM), erasable programmable read-only memory (EPROM), flash memory, or any other computer-readable storage media that is capable of storing program instructions or digital information.

The media used by persistent storage 908 may also be removable. For example, a removable hard drive may be used for persistent storage 908. Other examples include optical and magnetic disks, thumb drives, and smart cards that are inserted into a drive for transfer onto another computer-readable storage medium that is also part of persistent storage 908.

Network adapter 912, in these examples, provides for communications with other data processing systems or devices. In these examples, network adapter 912 includes one or more network interface cards. Network adapter 912 may provide communications through the use of either or both physical and wireless communications links. The programs imaging interface 122 in workstation 120 and myocardial image analysis module 132 in server 130 may be downloaded to persistent storage 908 through network adapter 912.

I/O interface(s) 914 allows for input and output of data with other devices that may be connected to workstation 120 and server 130. For example, I/O interface 914 may provide a connection to external devices 920 such as a keyboard, keypad, a touch screen, and/or some other suitable input device. External devices 920 can also include portable computer-readable storage media such as, for example, thumb drives, portable optical or magnetic disks, and memory cards. Software and data used to practice embodiments of the present invention, e.g., programs imaging interface 122 in workstation 120 and myocardial image analysis module 132 in server 130, can be stored on such portable computer-readable storage media and can be loaded onto persistent storage 908 via I/O interface(s) 914. I/O interface(s) 914 can also connect to a display 922.

Display 922 provides a mechanism to display data to a user and may be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

While steps of the disclosed method and components of the disclosed systems and environments have been sequentially or serially identified using numbers and letters, such numbering or lettering is not an indication that such steps must be performed in the order recited and is merely provided to facilitate clear referencing of the method's steps. Furthermore, steps of the method may be performed in parallel to perform their described functionality.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

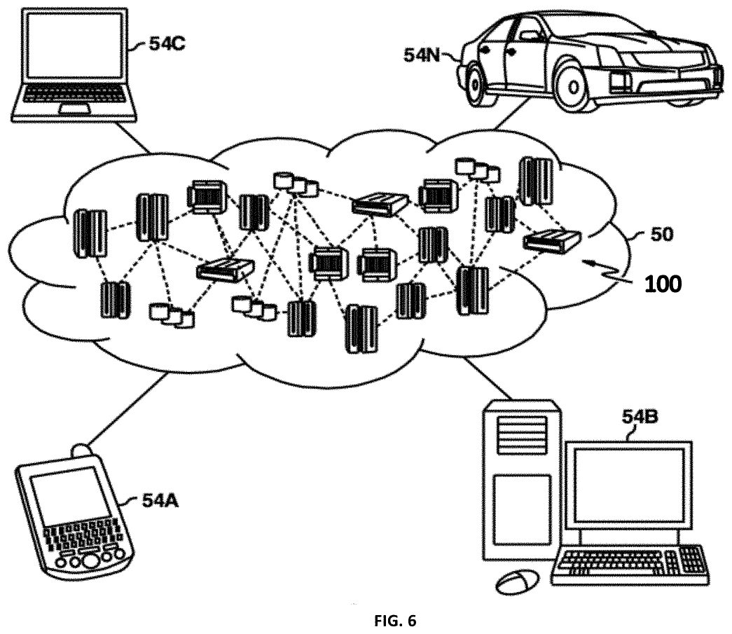

Referring now to FIG. 6, illustrative cloud computing environment 50 is depicted. As shown, cloud computing environment 50 includes one or more cloud computing nodes 100 with which local computing devices used by cloud consumers, such as, for example, personal digital assistant (PDA) or cellular telephone 54A, desktop computer 54B, laptop computer 54C, and/or automobile computer system 54N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 100 and cloud computing environment 50 can communicate with any type of computerized device over any type of network and/or network addressable connection (e.g., using a web browser).

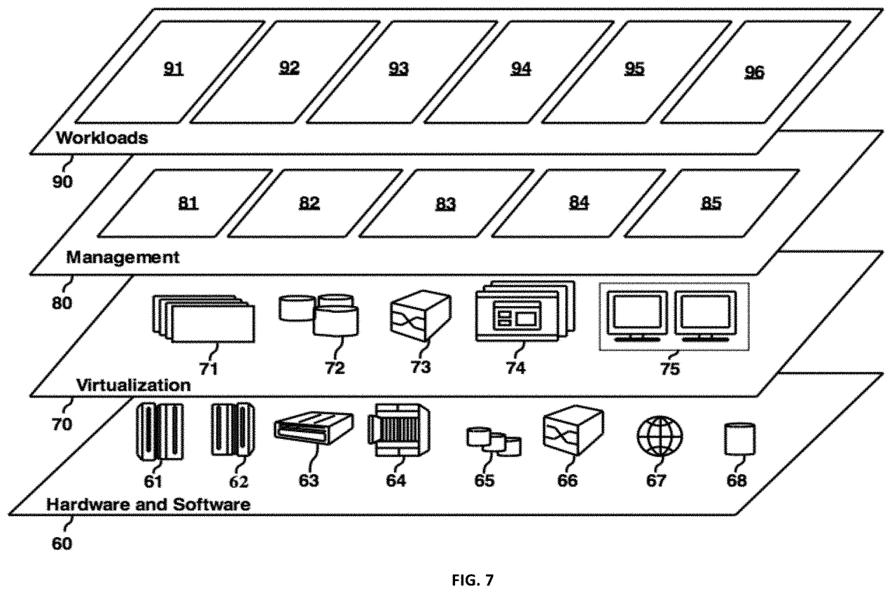

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and myocardium contraction detection environment 96. myocardium contraction detection environment 96 may relate perform a comparison of at rest and at stress representations of cardiac function (i.e., myocardium ultrasound images at rest and at stress) of a patient, acquired during a stress echocardiogram, and visually identify potential areas of myocardial ischemia.

The descriptions of the various embodiments of the present invention have been presented for purposes of illustration, but are not intended to be exhaustive or limited to the embodiments disclosed. The terminology used herein was chosen to explain the principles of the one or more embodiments, the practical application or technical improvement over technologies found in the marketplace, or to enable others of ordinary skill in the art to understand the embodiments. Various modifications, additions, substitutions, and the like will be apparent to those of ordinary skill in the art without departing from the scope and spirit of the invention, as defined in the following claims.

What is claimed is:

1. A computer-implemented method for analyzing cardiac function of a patient, the computer-implemented method comprising:
receiving a plurality of digital image representations of cardiac function at rest;
receiving a plurality of digital image representations of cardiac function at stress;
selecting a digital image representation of cardiac function at rest and a corresponding digital image representation of cardiac function at stress;
aligning the selected representation of cardiac function at rest with the selected corresponding representation of cardiac function at stress; and
identifying a difference between the selected representation of cardiac function at rest and the selected corresponding representation of cardiac function at stress based on a displayed overlay of the aligned representations.

2. The computer-implemented method of claim 1, wherein the selected representation of cardiac function at rest and the selected corresponding representation of cardiac function at stress are digital ultrasound images taken during an echocardiogram, and wherein the selected representation of cardiac function at rest is a digital ultrasound image of a myocardium at mitral level in sagittal view, and wherein the selected corresponding representation of cardiac function at stress is a digital ultrasound image of the myocardium at mitral level in sagittal view.

3. The computer-implemented method of claim 1, wherein the selected representation of cardiac function at rest and the selected corresponding representation of cardiac function at stress are selected based on a ranking for each being above a threshold level, and wherein the selected representation of cardiac function at rest and the selected corresponding representation of cardiac function at stress are each ranked using a machine learning model trained on optimal cardiac representations acquired from a plurality of patients across a variety of demographics.

4. The computer-implemented method of claim 1, wherein the selected representation of cardiac function at rest and the selected corresponding representation of cardiac function at stress are selected based on having a highest degree of matching representation orientation, anatomical view, representation clarity, and/or diagnostic quality as it pertains to a view of myocardium function having clear pixels and anatomical landmarks to match between at rest and at stress states of the myocardium.

5. The computer-implemented method of claim 1, wherein aligning the selected representation of cardiac function at rest with the selected corresponding representation of cardiac function at stress comprises using a corresponding electrocardiogram of the patient to synchronize on an R-wave of the corresponding electrocardiogram and using speckle tracking echocardiography in pixels of the selected representations displayed on a monitor.

6. The computer-implemented method of claim 1, wherein aligning the selected representation of cardiac function at rest with the selected corresponding representation of cardiac function at stress comprises using endocardial and epicardial contours of the patient's heart as references to align the selected representation of cardiac function at rest and the selected representation of cardiac function at stress at each portion of a cardiac cycle, and wherein the endocardial and epicardial contours of the patient's heart are detected as a result of delineation, via the use of tissue doppler imaging and observed tissue doppler speeds, between myocardium and surrounding tissue and segmentation by endocardium (inner wall) and epicardium (outer wall).

7. The computer-implemented method of claim 1, wherein the overlay of the aligned representations comprises an overlay of the selected representation of cardiac function at rest with the selected corresponding representation of cardiac function at stress and utilizes color coding within pixels of the selected digital image representation of cardiac function at rest and the selected corresponding digital image representation of cardiac function at stress to differentiate between an at rest response and an at stress response of the selected representation of cardiac function at rest and the selected corresponding representation of cardiac function at stress, respectively.

8. The computer-implemented method of claim 2, further comprising:
prompting, in real time, a medical ultrasound technician performing the echocardiogram of the patient during a stress phase on how to position an ultrasound probe so as to acquire at least one digital ultrasound image of the patient's heart at stress for correlation and/or comparison to at least one digital ultrasound image of the patient's heart at rest previously acquired during the echocardiogram.

9. A computer program product for analyzing cardiac function of a patient, the computer program product comprising:
one or more computer-readable tangible storage devices and program instructions stored on at least one of the one or more computer-readable tangible storage devices, wherein the program instructions are executable by a computer, the program instructions comprising:
program instructions to receive a plurality of digital image representations of cardiac function at rest;
program instructions to receive a plurality of digital image representations of cardiac function at stress;
program instructions to select a digital image representation of cardiac function at rest and a corresponding digital image representation of cardiac function at stress;
program instructions to align the selected representation of cardiac function at rest with the selected corresponding representation of cardiac function at stress; and
program instructions to identify a difference between the selected representation of cardiac function at rest and the selected corresponding representation of cardiac function at stress based on a displayed overlay of the aligned representations.

10. The computer program product of claim 9, wherein the selected representation of cardiac function at rest and the selected corresponding representation of cardiac function at stress are digital ultrasound images taken during an echocardiogram, and wherein the selected representation of cardiac function at rest is a digital ultrasound image of a myocardium at mitral level in sagittal view, and wherein the selected corresponding representation of cardiac function at stress is a digital ultrasound image of the myocardium at mitral level in sagittal view.

11. The computer program product of claim 9, wherein the selected representation of cardiac function at rest and the selected corresponding representation of cardiac function at stress are selected based on a ranking for each being above a threshold level, and wherein the selected representation of cardiac function at rest and the selected corresponding representation of cardiac function at stress are each ranked using a machine learning model trained on optimal cardiac representations acquired from a plurality of patients across a variety of demographics.

12. The computer program product of claim 9, wherein the selected representation of cardiac function at rest and the selected corresponding representation of cardiac function at stress are selected based on having a highest degree of matching representation orientation, anatomical view, representation clarity, and/or diagnostic quality as it pertains to a view of myocardium function having clear pixels and anatomical landmarks to match between at rest and at stress states of the myocardium.

13. The computer program product of claim 9, wherein aligning the selected representation of cardiac function at rest with the selected corresponding representation of cardiac function at stress comprises using a corresponding electrocardiogram of the patient to synchronize on an R-wave of the corresponding electrocardiogram and using speckle tracking echocardiography in pixels of the selected representations displayed on a monitor.

14. The computer program product of claim 9, wherein aligning the selected representation of cardiac function at rest with the selected corresponding representation of cardiac function at stress comprises using endocardial and epicardial contours of the patient's heart as references to align the selected representation of cardiac function at rest and the selected representation of cardiac function at stress at each portion of a cardiac cycle, and wherein the endocardial and epicardial contours of the patient's heart are detected as a result of delineation, via the use of tissue doppler imaging and observed tissue doppler speeds, between myocardium and surrounding tissue and segmentation by endocardium (inner wall) and epicardium (outer wall).

15. The computer program product of claim 9, wherein the overlay of the aligned representations comprises an overlay of the selected representation of cardiac function at rest with the selected corresponding representation of cardiac function at stress and utilizes color coding within pixels of the selected digital image representation of cardiac function at rest and the selected corresponding digital image representation of cardiac function at stress to differentiate between an at rest response and an at stress response of the selected representation of cardiac function at rest and the selected corresponding representation of cardiac function at stress, respectively.

16. The computer program product of claim 9, further comprising:
program instructions to prompt, in real time, a medical ultrasound technician performing the echocardiogram of the patient during a stress phase on how to position an ultrasound probe so as to acquire at least one digital ultrasound image of the patient's heart at stress for correlation and/or comparison to at least one digital ultrasound image of the patient's heart at rest previously acquired during the echocardiogram.

17. A computer system for analyzing cardiac function of a patient, the computer system comprising:
one or more processors, one or more computer-readable memories, one or more computer-readable tangible storage devices, and program instructions stored on at least one of the one or more computer-readable tangible storage devices for execution by at least one of the one or more processors via at least one of the one or more memories, the program instructions comprising:
program instructions to receive a plurality of representations of cardiac function at rest;
program instructions to receive a plurality of representations of cardiac function at stress;
program instructions to select a representation of cardiac function at rest and a corresponding representation of cardiac function at stress;
program instructions to align the selected representation of cardiac function at rest with the selected corresponding representation of cardiac function at stress; and
program instructions to identify a difference between the selected representation of cardiac function at rest and the selected corresponding representation of cardiac function at stress based on a displayed overlay of the aligned representations.

18. The computer system of claim 17, The computer-implemented method of claim 1, wherein aligning the selected representation of cardiac function at rest with the selected corresponding representation of cardiac function at stress comprises using endocardial and epicardial contours of the patient's heart as references to align the selected representation of cardiac function at rest and the selected representation of cardiac function at stress at each portion of a cardiac cycle, and wherein the endocardial and epicardial contours of the patient's heart are detected as a result of delineation, via the use of tissue doppler imaging and observed tissue doppler speeds, between myocardium and surrounding tissue and segmentation by endocardium (inner wall) and epicardium (outer wall).

19. The computer system of claim 17, The computer-implemented method of claim 1, wherein aligning the selected representation of cardiac function at rest with the selected corresponding representation of cardiac function at stress comprises using a corresponding electrocardiogram of the patient to synchronize on an R-wave of the corresponding electrocardiogram and using speckle tracking echocardiography in pixels of the selected representations displayed on a monitor.

20. The computer system of claim 17, further comprising:
program instructions to prompt, in real time, a medical ultrasound technician performing the echocardiogram of the patient during a stress phase on how to position an ultrasound probe so as to acquire at least one digital ultrasound image of the patient's heart at stress for correlation and/or comparison to at least one digital ultrasound image of the patient's heart at rest previously acquired during the echocardiogram.
